(19) United States
(12) Patent Application Publication
(10) Pub. No.: US 2006/0115894 A1
(43) Pub. Date: Jun. 1, 2006

(54) Method of and apparatus for facilitating processes of mammalian cells

(76) Inventor: Margaret Sin Ka Wan, Oxfordshire (GB)

Correspondence Address: Battelle Memorial Institute, 505 King Avenue, Columbus, OH, 43201-2693, US

(21) Appl. No.: 10/525,259

(22) PCT Filed: Aug. 14, 2003
(86) PCT No.: PCT/GB03/03558

§ 371 Date: Feb. 22, 2005

(30) Foreign Application Priority Data
Aug. 22, 2002 (GB) 0219618.6

(51) Int. Cl. C12N 5/08; C12M 3/00; C12N 5/06

(52) U.S. Cl. 435/325; 435/366; 435/289.1

(57) ABSTRACT

A method of facilitating processes of mammalian cells such as at least one of attachment, movement, growth, proliferation and differentiation comprises: supplying liquid comprising biologically compatible polymer to a liquid outlet in the vicinity of a surface and subjecting liquid issuing from the outlet to an electric field to cause the liquid to form polymer fibre which is attracted to and deposits onto the substrate to form a polymer fibre scaffold having fibre of a given diameter with gaps between adjacent fibre portions; and applying mammalian cells to the fibre scaffold, wherein the gaps between the fibre portions and the fibre diameter have a size relative to a diameter of the mammalian cells such that cells grow or elongate preferentially along the fibre of the fibre scaffold. Apparatus for enabling carrying out of such a method is also described.

This invention relates to a method of and apparatus for facilitating processes of mammalian cells with the aim of enabling formation of biological tissue for, for example, replacement of diseased or damaged natural tissue.

U.S. Pat. No. 5,041,138 describes methods of growing cartilaginous structures on biodegradable biocompatible fibrous polymer matrices formed by casting, compression molding, filament drawing or meshing while U.S. Pat. No. 6,228,117 describes various methods for facilitating bone tissue engineering that involve the use of a non-porous or partially or filly porous scaffold or three-dimensional matrix or film where porosity may be achieved as a result of ordered fibres, woven fibre meshes or open cell foams.

In one aspect, the present invention aims to provide a method of facilitating at least one cell process of mammalian cells, which method comprises using electric field effect technology to form a matrix or scaffold of biologically compatible polymer fibre having a fibre diameter and gap size between adjacent fibre portions that facilitates at least one cell process to enable formation of biological tissue.

In one aspect of the present invention liquid comprising a biologically compatible polymer is supplied to a liquid outlet and liquid issuing from the outlet is subjected to an electric field to cause the liquid to form polymer fibre which is deposited onto a target surface to form a scaffold or matrix comprising a three-dimensional continuous network of intercommunicating fibre or fibre portions, that is a network wherein fibre portions are interconnected and/or there are points or locations at which separation between the fibres or fibre portions is sufficiently small in relation to the size of cells to be applied to the fibre scaffold, that the cells respond as if the fibres or fibre portions were physically connected at those points or locations, wherein the fibre diameter and a gap size between fibre portions is controlled to facilitate at least one cell process to enable formation of biological tissue.

In one aspect, there is provided a porous biologically compatible, biodegradable and/or bioresorbable fibrous polymeric scaffold or matrix, generated by electric field effect technology (EFET), for facilitating at least one at least one cell process and formation of tissues such as bone, ligament, cartilage and tendon.

The at least one cell process may be any of attachment, movement, growth, proliferation and differentiation.

In embodiments, the fibre gap is greater than approximately half the cell diameter.

In embodiments, the fibre diameter is less than the fibre gap.

The polymer formulation may comprise a polymer solution. Where this is the case, the fibre diameter may be in the range from 0.2 to 100 microns while the gap size may be in the range from about 10 to 500 microns.

The polymer formulation may comprise a polymer melt. Where this is the case, the fibre diameter may be in the range from 2 to 500 microns while the gap size may be in the range from about 25 to 3000 microns.

The relative sizes of the cell and fibre diameters may be such that the fibre surface appears curved to the cells and, for example, the fibre diameter may be of comparable size to cell surface receptors of the cells.

In embodiments, the cell diameter is from 1 to 20 times greater than the fibre diameter. For example, in one embodiment, the cell diameter is from 5 to 10 times greater than the fibre diameter.

In embodiments, the fibre diameter is in the range from about 1 to 2 microns. For example, in an embodiment the cell diameter is about 10 microns and the fibre diameter is from 1 to 2 microns.

In embodiments, the polymer is selected from the group consisting of New Skin, Eudragit RL100, polycaprolactone (PCL-65), polylactide (L:D isomer ratio 50:50) and polylactide (L:D isomer ratio 96:4).

In embodiments, the cells are human cells such as fibroblast cells, for example human skin fibroblast cells or human bone marrow fibroblast cells. In embodiments the cell may be stem cells that can be encouraged to differentiate by the fibre of the fibre scaffold.

In some embodiments the fibre scaffold may be formed in vitro. Such fibre scaffolds may be arranged to be implanted in a mammalian body or placed on or in a wound. In other embodiments the surface or substrate is a target area of a mammalian body such as a wound and the fibre scaffold is produced in situ.

In embodiments the cells are applied by a seeding process. In other embodiments the cells may be applied by spraying.

In one aspect, the present invention provides a method of forming a polymer fibre scaffold, for example to form a wound dressing, which method comprises producing polymer fibre using electric field effect techniques so that the polymer fibre deposits onto the surface of a target area, such as skin and/or wound, to form a covering or dressing for the target area, wherein the polymer fibre production is controlled to control the polymer charge and relaxation time, and thereby control the lateral force experienced by the polymer fibre resulting from the fibre that has already settled on the target area, so as to control the pattern of deposition of the polymer fibre on the target area, to produce a lattice or web like polymer fibre scaffold to facilitate the formation of skin tissue by fibroblasts of a weave pattern rather than an aligned parallel pattern.

As used herein, the term “biologically compatible polymer” means that the polymer is compatible with the mammalian cell and/or body with which the polymer fibre scaffold is intended to come into contact. The polymer fibre scaffold may lose its structural integrity over time by, for example, at least partially disintegrating or dissolving into or being absorbed by the environment in which it is placed so that the fibre scaffold structure disappears after having served its purpose as a scaffold for the formation of biological tissue or precursors thereto, as the case may be. For example, the polymer may be “biodegradable”, that is the polymer may degrade so that the fibre scaffold disintegrates over time when used in the manner intended (for example so that the fibre scaffold structure disappears after having served its purpose as a scaffold for the formation of biological tissue or precursors thereto, as the case may be) or may be “bioresorbable”, that is the polymer may be absorbed into the surrounding environment over time, so that the fibre scaffold structure disappears after having served its purpose as a scaffold for the formation of biological tissue or precursors thereto, as the case may be. As used herein, the term “electric field effect technology” or “EFET” means a technology that uses the effect of an electric field on liquid to cause the liquid, depending upon the process conditions and liquid formulation, to form fibre, droplets, particles or fibre segments (“fibrils”), for example as discussed in WO98/03267, the whole contents of which are hereby incorporated by reference.

Embodiments of the present invention will now be described, by way of example, with reference to the accompanying drawings, in which:

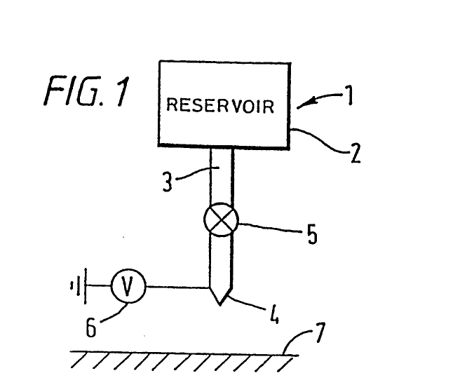

FIG. 1 shows very diagrammatically one example of apparatus suitable for use in a method embodying the invention;

FIG. 2 shows very diagrammatically another example of apparatus suitable for use in a method embodying the invention;

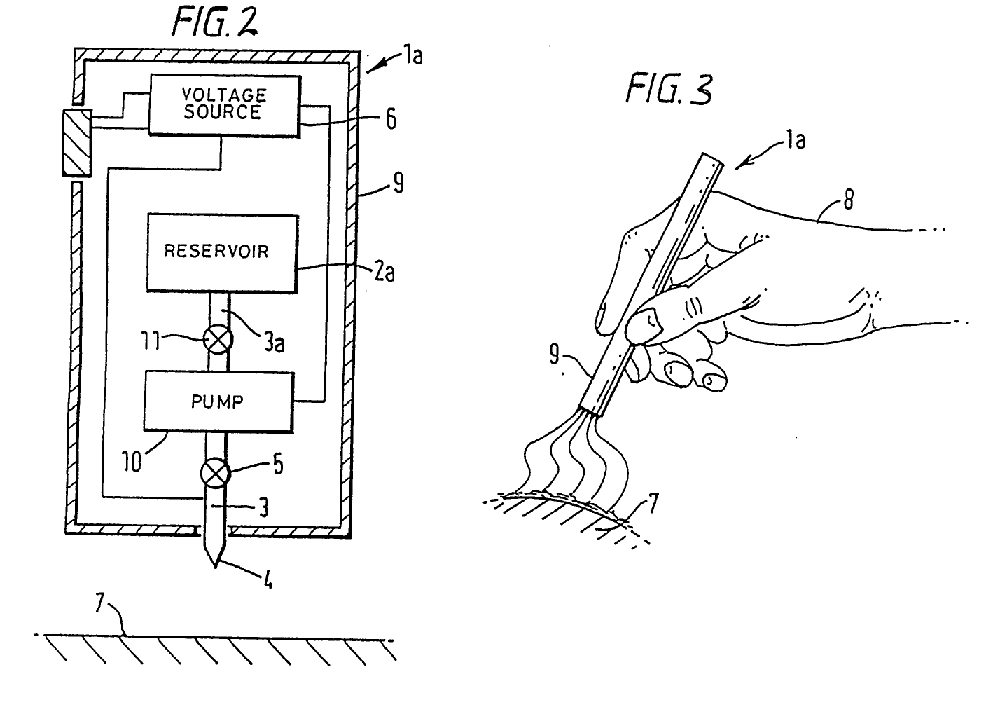

FIG. 3 shows schematically use of the apparatus shown in FIG. 2 to form a polymer fibre scaffold on a surface area;

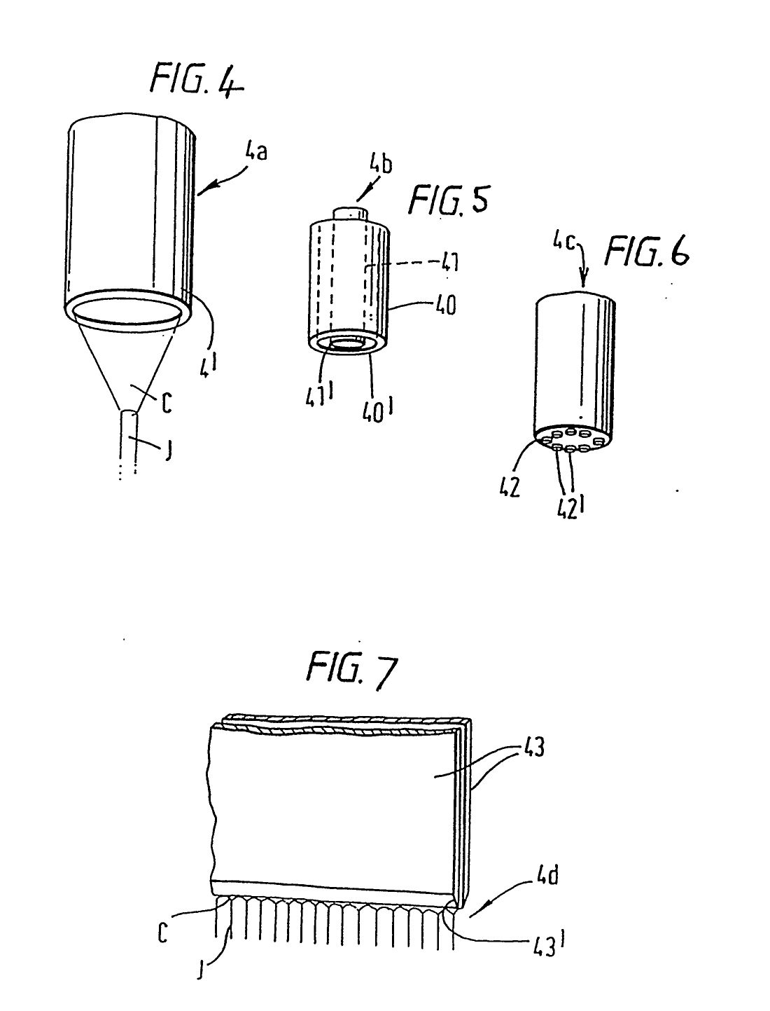

FIGS. 4 to 7 illustrate various different types of nozzles or outlets for apparatus such as that shown in FIG. 1 or 2;

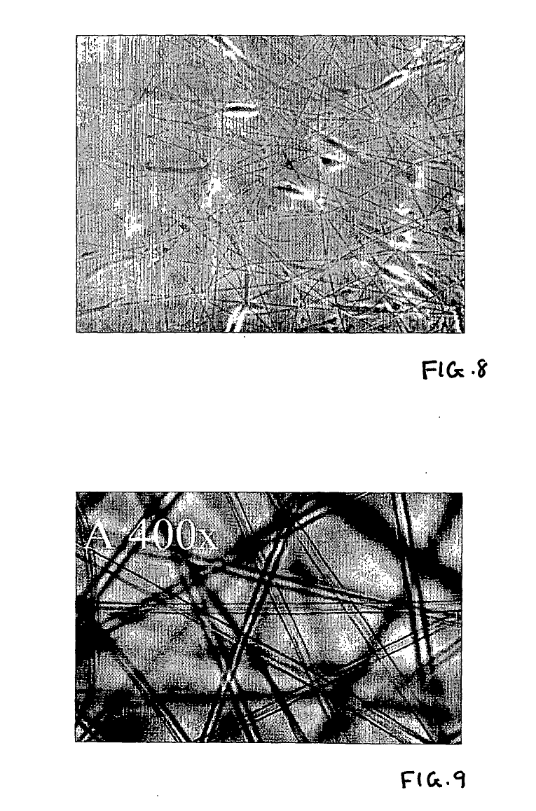

FIG. 8 shows a reproduction of a photograph originally taken at 200 times magnification illustrating growth of human fibroblast cells along a polymer fibre scaffold or matrix formed of polylactide (L:D isomer 96:4);

FIG. 9 shows a reproduction of a photograph taken at 100 times magnification illustrating a polymer fibre scaffold or matrix formed of Eudragit E100.

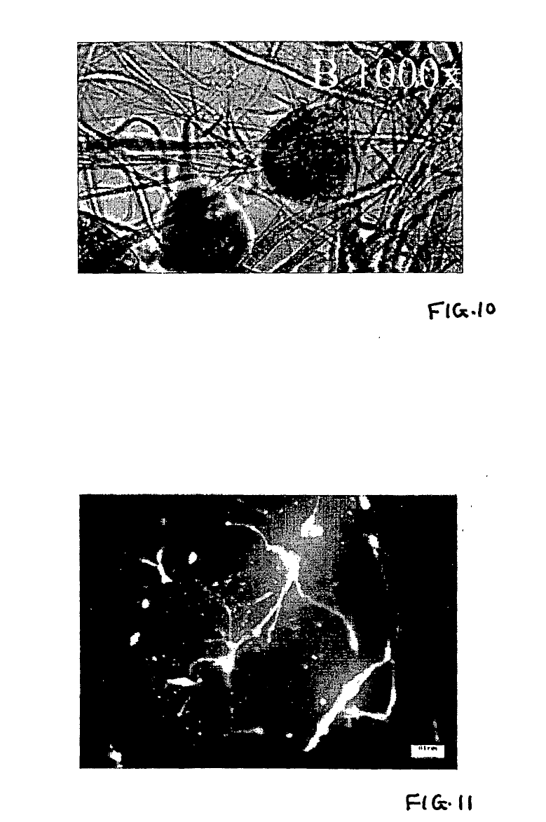

FIG. 10 shows a reproduction of a photograph taken at 1000 times magnification illustrating the structure formed by growth of cells on a PCL-65 polymer fibre scaffold or matrix for seven days;

FIG. 11 shows a reproduction of a photograph taken at 100 times magnification illustrating cell growth of green fluorescent protein-labelled human bone marrow fibroblast cells (HBMF cells) on a PCL-65 polymer fibre scaffold after 7 days in cell culture medium; and

FIG. 12 shows a reproduction of a focused ion beam scan, at 1000 times magnification, of HBMF cells on a PCL-65 polymer fibre scaffold or matrix after seven days in cell culture medium.

Referring now to the drawings, FIG. 1 which shows very diagrammatically one example of apparatus 1 suitable for forming a polymer fibre scaffold or matrix on a surface area 7 for facilitating at least one of the cell processes of cell attachment, movement, growth, proliferation and differentiation to enable formation of biological tissue.

The apparatus 1 comprises a reservoir or container 2 for containing a biologically compatible polymer formulation. The reservoir 2 is coupled via a liquid supply pipe 3 and a flow regulating valve 5 to a liquid outlet or nozzle 4 to which a voltage is applied by a voltage source 6 by means of a switch (not shown). The flow regulating valve 5 may be a user-operable mechanical valve or an electrically operable valve, for example. The voltage source 6 is arranged to provide a high voltage sufficient to enable generation of an electric field strong enough to cause a liquid polymer formulation issuing from the outlet 4 to form at least one fibre-forming jet when subjected to the electric field. Typically, the voltage source is arranged to provide a voltage in the range of 15 to 25 kV to liquid issuing from the outlet 4.

In order to produce a three-dimensional polymer fibre network or scaffold suitable for forming a polymer fibre scaffold or matrix for facilitating at least one of the cell processes of attachment, movement, growth, proliferation and differentiation to enable formation of biological tissue using either the apparatus 1, the user first places a biologically compatible liquid polymer formulation within the container or reservoir 2 of the apparatus 1 and positions the apparatus 1 so that the outlet 4 of the liquid supply pipe 3 is a few centimetres, for example from 5 to 10 cm, above the earthed or grounded surface area 7 onto which the polymer fibre matrix is to be formed.

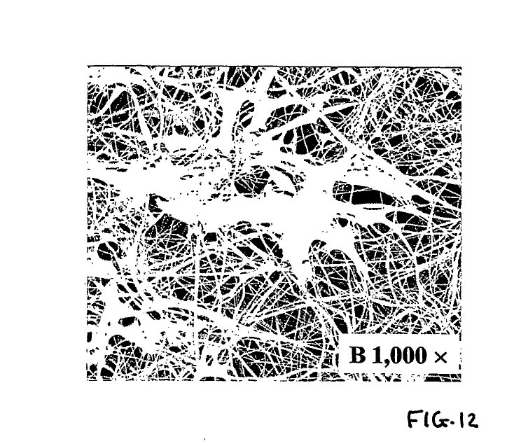

The user then activates a switch (not shown in FIG. 1) to connect the voltage source 6 and operates the flow regulating valve 5 to cause liquid to be supplied under gravity from the reservoir 2 through the liquid supply pipe 3 to the outlet 4 at a required flow rate. The polymer formulation issuing from the outlet 4 is subjected to a high electric field generated by the voltage source. This electric field which causes the polymer formulation to form a cone and at least one jet which, before it can be separated by the applied electric field into liquid droplets, at least partially solidifies in flight to form an electrically charged fibre. The electrically charged fibre moves towards and deposits onto the surface area 7 where it loses its electrical charge and forms a three-dimensional network or scaffold of interconnected polymer fibre. Depending upon the polymer formulation, time of flight and environmental factors, the fibre may dry or solidify during flight, or may be partially solid, gel-like or possibly even still at least partially liquid at the time of deposition on the surface. The state of the fibre can be controlled for a given polymer formulation by, for example, adjusting at least one of the time of flight (by changing the separation between the outlet 4 and surface 7) and the rate of evaporation of solvent where the polymer formulation is a solution (for example by control of at least one of the environmental temperature and vapour pressure of the polymer solvent) with a longer time of flight and/or a higher rate of solvent evaporation resulting in a fibre that is drier when it deposits on the surface and vice versa. Examples of the structure of the polymer fibre scaffold or network are shown by the reproductions in FIGS. 11 to 15 of photographs produced during experiments which will be discussed in greater detail below.

The apparatus 1 shown in FIG. 1 uses a gravity feed to supply polymer formulation to the outlet 4. This has the advantage of simplicity. FIG. 2 illustrates a part cross-sectional view of another form of apparatus 1 a suitable for use in a method embodying the invention. The apparatus 1a is, as illustrated schematically in FIG. 3, intended to be portable, in particular to be held in the hand 8 of a user, and does not rely on gravity feed.

The apparatus 1a comprises a housing 9 within which is mounted a reservoir 2a of the polymer formulation to be dispensed. The reservoir 2a may be formed as a collapsible bag so as to avoid any air contact with the liquid being dispensed. The reservoir 2a is coupled via a supply pipe 3a to a pump chamber 10 which is itself coupled via the supply pipe 3 and the flow regulating valve 5 to the outlet 4 in a similar manner to that shown in FIG. 1. The voltage source 6 in this example is coupled to a user-operable switch SW1 which may be a conventional push button or toggle switch, for example. The voltage source 6 may comprise, for example a piezoelectric high voltage source of the type described in WO94/12285 or a battery operated electromagnetic high voltage multiplier such as that manufactured by Brandenburg, ASTEC Europe of Stourbridge West Midlands, UK or Start Spellman of Pulborough, West Sussex, UK and typically provides a voltage in the range of from 10 to 25 kV. Although not shown, a voltage control circuit comprising one or more resistor capacitor networks may be provided to ramp the voltage up smoothly.

The reservoir 2a may be coupled to the pump chamber 10 by way of a valve 11 which may be a simple user-operable non-return or one way valve or may be an electrically or mechanically operable valve of any suitable type, for example a solenoid or piezoelectric valve, operable by a voltage supplied by the aforementioned control circuit.

The pump chamber 10 may comprise any suitable form of pump, which provides a continuous substantially constant flow rate, for example an electrically operable pump such as a piezoelectric, or diaphragm pump or an electrohydrodynamic pump as described in EP-A-0029301 or EP-A-0102713 or an electroosmotic pump as described in WO94/12285 or a mechanically operable pump such as syringe pump operated or primed by a spring biassing arrangement operable by a user.

FIGS. 4 to 7 illustrate schematically some examples of outlet or nozzle 4 that may be used in the apparatus shown in FIGS. 1 and 2 and 3. The nozzle 4a shown in FIG. 4 comprises a hollow cylinder which is formed of electrically conductive or semiconductive material at least adjacent its end 4′ where the voltage is to be applied in use and will in use produce one or more jets (one cusp or cone C and jet J are shown) depending upon the resistivity and flow rate of the polymer formulation and the voltage applied to the outlet 4. The nozzle 4b shown in FIG. 5 comprises two coaxial cylinders 40 and 41 at least one of which is electrically conductive or semiconductive at least adjacent its end 40′ or 41′ where the voltage is applied and will in use produce a number of jets depending upon the resistivity and flow rate of the polymer formulation and the applied voltage. The nozzle 4c shown in FIG. 6 comprises a number of parallel capillary outlets 42 which electrically conductive or semiconductive at least adjacent their ends 42′ where the voltage is applied. Each capillary outlet 42 will normally produce a single jet. The multiple nozzles shown in FIG. 6 have the advantage that blockage of one nozzle by relatively viscous polymer formulation does not significantly affect the operation of the device and also allow different polymer formulations to be supplied from respective reservoirs to different ones of the nozzles, if required. The nozzle 4d shown in FIG. 7 comprises a slot-shaped nozzle defined between two parallel plates 43 which are electrically conductive or semiconductive at least adjacent their ends 43′ where the voltage is applied. The use of a slot nozzle when relatively highly viscous polymer formulations are being used is advantageous because complete blockage of the nozzle is unlikely, as compared to the case where a relatively fine capillary nozzle is used, and a partial blockage should not significantly affect the functioning of the device because the polymer formulation should be able to flow round any such partial blockage. The use of a slot-shaped nozzle outlet as shown in FIG. 7 also allows a linear array of jets and thus of fibres to be formed. Where the polymer formulation being used is sufficiently conductive to enable the voltage to be applied to the polymer formulation rather than the nozzle then the nozzle may be formed of any suitable electrically insulative material which does not retain electrical charge for any significant length of time, for example glass or a semi-insulating plastic such as polyacetyl. Another possibility is the fibre comminution site or nozzle described in WO95/26234.

In use of the apparatus 1a shown in FIGS. 2 and 3, the user first positions the apparatus over the earthed (grounded) surface area 7 on which the polymer fibre network or scaffold is to be formed, then actuates the switch SW1 and the pump of the pump chamber 10 to cause, when the valves 5 and 11 are opened, a stream of polymer formulation to be supplied to the outlet 4 where the polymer formulation is subjected to the applied electric field resulting in formation of at least one jet which forms electrically charged fibre which is attracted to and deposits onto the surface area 7 to form a three-dimensional polymer fibre network or scaffold on the surface area 7 as described above with reference to FIG. 1. The user may move the apparatus 1a relative to the area 7 to cause the fibre scaffold to cover a larger area.

In operation of either the apparatus 1 or la described above, the polymer fibre deposits onto the surface area 7 swiftly, uniformly and gently by the energy contained in the electric field used to generate the fibre and does not over spray, nor become trapped in air streams and swept away from the surface area.

As mentioned above, FIGS. 11 to 15 show typical examples of the pattern of deposition of fibre forming the fibre scaffold. It is believed that these patterns result because, as the polymer fibre deposits on the surface area, immediately after a part of the fibre touches the surface area 7, the remaining fibre experiences a lateral force, due to repulsion of the fibre that has settled on the surface area 7 but has not lost its electrical charge. If so, the degree of the lateral force will be related to the amount of electrical charge on the settled (non-moving) part of the fibre being laid down, and this is inversely proportional to the relaxation time of the polymer (that is the time to lose its electrical charge which may itself be related to the dielectric constant and resistivity of the polymer fibre) so that, when the polymer's relaxation time is short, say a microsecond, a small lateral force will be developed on the moving fibre; while when the polymer's relaxation time is long a large lateral force will be developed on the moving fibre. The electrical charge on the fibre and thus the relaxation time may be adjusted by bombarding the fibre and/or scaffold with gaseous ions by using gaseous ions of the same polarity to increase the lateral forces and gaseous ions of the opposite polarity to reduce the lateral forces. The lateral movement of the fibre may be controlled or adjusted as described above by effecting relative movement between the surface area 7 and the outlet 4 to enable coverage of a large surface area.

The thickness of the polymer fibre scaffold or matrix is limited by the repulsive forces exerted as more fibre attempts to settle on to the already formed fibre scaffold and is therefore controlled by the polymer fibre relaxation time. A relaxation time of say a few milliseconds should exert useful lateral forces in order to move the settling fibre, but should also quickly allow the fibres to return and settle, so as to enable a multi-layer scaffold of fibres to be formed.

The fibre diameter and fibre gap size (that is the average separation of adjacent fibre portions) of the polymer fibre network or scaffold deposited on the surface area 7 are determined by the fibre production parameters which include the applied voltage, the viscosity and resistivity of the polymer formulation and the resultant polymer fibre, the drying rate of the polymer formulation (that is the evaporation rate in the case of a polymer solution), and the mass flow rate of the jet or jets, the mass flow rate for a given polymer formulation being determined by the liquid flow rate. Accordingly, the fibre diameter and fibre gap size of the fibre scaffold can be controlled by controlling theses fibre production parameters.

The polymer fibre scaffold may be designed to lose its structural integrity over time by, for example, at least partially disintegrating or dissolving into or being absorbed by the environment in which it is placed so that the fibre scaffold structure disappears after having served its purpose as a scaffold for the formation of biological tissue or precursors thereto, as the case may be. For example, the polymer may be a biodegradable or bioresorbable polymer.

As will be described in greater detail below, the polymer formulation may, depending upon the characteristics of the polymer, comprise a polymer solution or a polymer melt. Where the polymer formulation is a polymer solution then, the resulting fibre scaffold will typically have a fibre diameter in the range from 0.2 to 100 microns and a fibre gap size in the range from about 10 to 500 microns. Where the polymer formulation is a polymer melt, then, typically, the fibre diameter will be in the range from 2 to 500 microns while the gap size will be in the range from about 25 to 3000 microns.

Experiments have been carried out in which polymer fibre scaffolds or networks produced as described above have been seeded or sprayed with various types of mammalian cells, including human cells with the aim of enabling or facilitating formation of biological tissue. These experiments have shown the importance of control over and appropriate selection of the fibre diameter and fibre gap of the polymer fibre scaffold or matrix for facilitating at least one of the cell processes of cell attachment, movement, growth, proliferation and differentiation for these cells to enable formation of biological tissue.

Thus the fibre gaps have been found to provide a lattice of internal space through which cell culture medium, biologically active factors, nutrients and gas can be supplied to the internal parts of the fibre scaffold, and by-products can diffuse out from the fibre scaffold and have been found to be conducive to cell attachment and maintenance of cell function. It has also been found that the fibre gap size required for the fibre scaffold appears to be cell type dependent, and may be important for cell movement, differentiation, growth, proliferation, neo-vascularisation and production of extracellular matrix.

As regards fibre diameter, as a result of the experiments, it is believed that, if the fibre diameter is of comparable, smaller dimension to the cell, a signal to grow in a preferred direction, that is along the fibre, is established. The fibre diameter, together with the polymer's surface chemistry and topography, are also believed to affect the signal that may accelerate or decelerate the growth rate and cell differentiation. The cell diameter may be from 1 to 20 times the fibre diameter. For example, the cell diameter may be from 5 to 10 times greater than the fibre diameter. A fibre diameter between 1 and 10 microns may be optimal for growing skin fibroblasts and forming skin tissue, and for progenitor stem cells differentiating into another cell type without the addition of extrinsic proteins such as growth factors, and for cell proliferating in preferred growth rates.

The fact that the experiments show that cells appear migrate to and move along the fibres of the polymer fibre network or scaffold may have particular advantages in the area of skin regrowth. Thus, scar formation is believed to be due to an evolutionary action of cytokines that causes new skin to be formed as quickly as possible, to prevent infection. Parallel, rather than interwoven patterns, can be shown to provide the quickest way of forming new skin, however such parallel patterns tend to produce scar tissue. If, however, skin fibroblasts can be caused to migrate to and move along the fibre of the polymer fibre scaffold, then the lattice or network-like formation of fibres (as illustrated by the photographs shown in FIGS. 8 to 12) may be thus used to make fibroblasts form a weave pattern, rather than the aligned, parallel pattern that produces scar tissue. The fibroblasts should at the same time lay down a collagen basal layer for the next layer of skin to start the full process of tissue repair, therefore reducing the scar formation.

Details of examples of the experiments discussed above are set out below.

### EXAMPLE 1

In this example, the apparatus 1 shown in FIG. 1 was used to generate different polymer fibre scaffolds or networks of 0.16-0.19 mm thickness on 22×22 mm glass coverslips which thus provided the substrate or surface area 7. The fibre production parameters were controlled to produce different diameter fibres. The different polymers used were:
polymer 1: New Skin (trade mark)
polymer 2: Eudragit (trade mark) RL100
polymer 3: polycaprolactone of molecular weight 65,000 (CL-65),
polymer 4: polylactide (L:D isomer=50:50),
polymer 5: polylactide (L:D isomer=96:4)

New Skin (trade mark) is marketed by SmithKline Beecham and comprises nitrocellulose in an organic solution (in particular it comprises ethyl acetate, isopropyl alcohol, amyl acetate, isobutyl alcohol, denatured alcohol, camphor and nitrocellulose) while Eudragit (trade mark) RL100 is marketed by Röhm GmbH of Darmstadt, Germany.

Table 1 below shows the fibre production parameters used in this example. Glass coverslips without any fibres deposited thereon were used as controls.

TABLE 1

| | New | Eudragit | | Polylactide | Polylactide |
|---|---|---|---|---|---|
| | Skin | RL100 | PCL65 | (50:50) | (96:4) |
| Polymer | As | 25% in | 18.33% in | 3.33% in | 1.39% in |
| Formulation | sup- | ethyl | acetone | acetone | acetone |
| | plied | alcohol | | | |
| Nozzle to | 14 | 20 | 16.5 | 19 | 15.5 |
| surface | | | | | |
| area | | | | | |
| distance | | | | | |
| (cm) | | | | | |
| Polymer | 4 | 3 | 12 | 8 | 12 |
| formulation | | | | | |
| flow rate | | | | | |
| (ml/hr) | | | | | |
| Voltage (kV) | −22 | −20 | −28.5 | −15 | −15 |
| Fibre | 1-2 | 1-2 | 5 | 1-2 | 1-2 |
| Diameter | | | | | |
| (micron) | | | | | |

Once the polymer fibre scaffolds had been deposited onto the glass coverslips, these polymer fibre-coated coverslips were sterilised with beta-irradiation at AEA Technology, Oxford, England. The plain (that is the coverslips not coated with fibre scaffolds) coverslips were sterilised in 70% ethanol and then flame-dried before use. The polymer fibre coated-coverslips were pre-wet in phosphate to decrease the surface tension, before cells were seeded (3×10⁴ per coverslip) on the polymer fibres.

The following different types of cells were used:
cell type 1: human skin fibroblasts
cell type 2: Chinese Hamster Ovary cells (CHO)
cell type 3: SV40-transfected African Green monkey kidney cells
cell type 4: human epitheloid carcinoma of the cervix (HeLa)
cell type 5: a human histiocyte lymphoma cells (U937) (these cells are non-adherent)

Chemical Nature of Fibre Surfaces

It is thought that adhesion of cells to a surface is largely dependent on the chemical structure of a surface. As a preliminary experiment to determine the effect of different substrates on cell adhesion, respective coverglasses coated with the different polymer fibre scaffolds were put in a 150 mm-culture dish. Chinese Hamster Ovary (CHO) cells were seeded on top of the fibres and the dish.

Measurement of Cell Proliferation

Proliferation or metabolism of human skin fibroblasts was measured using a [3-(4,5-dimethylthiazol-2-yl)-2,5-diphenyltetrazolium bromide] (MTT) assay. The MTT assay measures the amount of an enzyme succinate dehydrogenase, SDH (a stable cytosolic enzyme that is released upon cell lysis), which converts the tetrazolium salt into an insoluble purple-blue formazan product. The absorbance of each sample was then read at 570 nm, and the intensity of the purple-blue colour that appears should be directly proportional to the number of viable cells.

Cell Morphology

The morphology of the cells grown on the polymer fibres was examined under light and phase contrast microscopes.

Results

Chemical Nature of Fibre Surfaces

The results obtained showed that the plating density (the number of cells settled down per unit area) on all the surfaces appear to be similar, implying that cells do not have a preference for a surface chemical structure.

Cell Adhesion

On the coverslips without polymer fibres, it was observed that the human skin fibroblasts adhered to the surface of the coverslips, and their processes spread completely. They also tended to form close parallel arrays as they approached confluence.

In contrast, on the coverslips coated with polymer fibre scaffold, all of the adherent cells (cell types 1 to 4) were found to attach to and align with the fibres of polymer 1, 2, 4 and 5. Also, these cells did not migrate to the glass space in between the polymer fibres. Fibres prepared from polymer 5 appeared to be the best substrate, as all of the adherent cell types (cell types 1 to 4) showed good growth along these fibres. On the contrary, only a few cells were found adhered to fibres of polymer 3 and most of them grew on the glass space in between the fibres. This indicates that adherent cells preferred to use the thinner diameter fibres of the polymer 1, 2, 4 and 5 fibre scaffolds as substrates.

FIG. 8 shows a reproduction of a photograph (taken at 200 times magnification) illustrating cell growth of human fibroblasts along a polymer fibre scaffold or matrix formed of polymer 5, that is polylactide (L:D isomer=96:4).

In this example, the fibres of polymer 3 (polycaprolactone) had a fibre diameter of 5 microns while the fibres of the other polymers had a fibre diameter of 1-2 microns. The fact that few cells adhered to the polymer 3 fibres indicates that the diameter of a fibre may be a key factor for cell adhesion and growth. Better cell growth with polymer 3 should occur where the fibres are of 1-2 micron diameter.

One reason for the cells preference for the thinner fibre is that it is possible that cells may be able to recognise fibres of a small diameter, such as 1-2 microns, as a curved surface and attach to them. This hypothesis is consistent with the findings that stronger growth of cells was found on a rough surface that was prepared by painting with polymer solutions than on an uncoated coverglass surface.

The non-adherent human histiocyte lymphoma cells (U937) (cell type 5) were used as a control cell line. The results for these non-adherent cells showed that these cells continued proliferating and did not adhere to the fibres of any of the fibre scaffolds.

Results from a preliminary experiment showed that CHO cells adhered to those fibres hanging over the edges of the coverslips. These hanging fibres are especially prevalent with polymer 1 and 3. However, most of the cells crawled through the larger diameter polymer 3 fibre mat on coverslips and grew preferentially on the surface of the coverslips.

It is known that adhesion proteins play an important role during cell adhesion, for example, L-selectins on the lymphocytes surface specifically bind to carbohydrates on the lining of lymph node vessels. Protein molecules present in human serum may therefore help binding of cells to the polymer fibres. To test this idea, fibre-coated coverglasses were submersed in normal human serum and incubated at 37° C. Proteins were extracted from the fibres and analysed on a polyacrylamide gel electrophoresis system. In order to visualise any protein bands present, the gel was stained with a very sensitive dye—silver nitrate. Interestingly, it was found that a protein of about 20 kD bound to the polymer fibres. The identity of the proteins remains to be determined by protein sequencing.

Cell Proliferation

The cells on all the fibre scaffolds grew over a period of about 14 days and gradually became sub-confluent, indicating that cell proliferation had occurred. For the fibre scaffolds seeded with human skin fibroblasts, proliferation of cells was confirmed by the MTT assay which showed that the purple-blue colour increased over a period of seven days, indicating that the polymer scaffold or mat provides a biological substrate to which cells can adhere and grow.

Cell Morphology

All the adherent cells (cell types 1 to 4) grew on the five different polymer type fibre scaffolds or mats at a similar rate, and no signs of cell lysis and toxicity were identified. They also had normal morphological characteristics when examined using light and phase contrast microscopes.

Normal Cell Morphology

In this study, we have demonstrated that human fibroblasts proliferated on polymer fibre mats prepared from polymers 1, 2, 4 and 5 (as did the other adherent cells tested). The cells showed normal morphology, and no evidence of cytotoxicity was detected.

Shape of Fibres

Apart from the chemical structure, it is increasingly being realised that the surface topography, especially on a fine scale, plays a vital role in the attachment of cells. Our results showed that cells preferred to elongate in the direction of a fibre. This finding was confirmed with results using CHO cells on fibres of polymers 1 (Newskin) and 3 (polycaprolactone).

Size of Fibres

Cell diameters are typically in the range of 2-20 microns; for example fibroblasts have a diameter of about 10 microns. Cells also have membranes with thickness of about 100 nm and cells surface receptors (10-100 nm) that control the interactions with their substrates. A possible reason for the adherent cells finding ways to adhere to and showing good growth along the 1-2 micron diameter polymer fibres is therefore that cells have a preference for attaching to surface features that are about the same size as that of a cell receptor. Also, because these fibres were of a small diameter, the cells may be able to recognise they are on a curved surface. Also, this curved surface may appear to the cells to be of similar shape to part of an adhesion molecule.

It is possible that the adherent cells may also prefer fibres having diameters smaller than 1-2 microns, for example, 10-100 nm (nanometres). However, whether cells grow along the fibres depends on the size of cells, the size of fibres and the gap size of the fibre scaffolds and, if the cells are very large compared to the size of the fibres and the gap size of the scaffolds, the cells may simply not “see” the individual fibres but will respond to the fibre scaffold as if it is effectively a membrane and will tend to adhere to the top of a few fibres with no cell migration occurring.

Signal Sent to Cells

One explanation for how the cells may “know” about the dimensions of the fibres substrates is as follows. A cell membrane has a close interaction with the internal cytoskeleton. The cytoskeleton is composed of actin microfilaments, intermediate filaments and microtubules, which give shape to a cell, provide support for cell extensions, and are involved in cell movement and interactions with the substratum on which the cell is lying. Any change in the substrate, for example, the weave pattern of the fibre scaffolds, will affect the generation of signals within the cell and cause some kind of activation process that results in the changing of cell shape.

Conclusions

Taken together, the mammalian adherent cells tested, including the human skin fibroblasts, preferred to adhere to and grow on polymer fibres of diameter of 1-2 microns rather than the 5 micron diameter fibres. It appears that the diameter of a fibre plays a key role in cell adhesion and growth, and acts as a physical means for cell signalling, as it may activate appropriate signals within cells and cause some kind of activation process that results in the changing of cell shape. This process could also be enhanced in the presence of some adhesion proteins.

### EXAMPLE 2

In this example different diameter fibre scaffolds of approximately 1 mm in thickness were formed from three different polymers: polycaprolactone with molecular, weight 65,000 (PCL-65); Eudragit E100 a polymer marketed by Röhm GmbH (and being a copolymer based on (2-Dimethylaminoethyl) methacrylate, butyl methacrylate and methyl methacrylate having a mean molecular weight of about 150000); and polymethymethcrylate (PMMA). The apparatus shown in FIG. 1 was again used but this time with aluminium foil as the surface area or substrate 7.

Table 2 below shows the fibre scaffold production parameters and the fibre diameter and fibre gap size of the resultant fibre scaffolds as determined using light and electron microscopes.

TABLE 2

| | PCL65 | Eudragit E100 | PMMA |
|---|---|---|---|
| Formulation | 20% in | 40% in ethyl | 25% in |
| | acetone | alcohol (weight | acetone |
| | (w/v) | by volume (w/v) | (w/v) |
| Nozzle to plate distance | 10 | 22 | 5 |
| (cm) | | | |
| Polymer formulation | 10 | 20 | 6 |
| flow rate (ml/hr) | | | |
| Voltage (kV) | 10 | 20 | 11 |
| Fibre diameter (micron) | 3 | 7.5 | 10 |
| Fibre gap size (micron) | 16 | 50-200 | 32 |

Once formed, the fibre scaffolds were removed from the aluminium foil for cell culturing and the biological compatibility of the scaffolds was tested by seeding and growing human bone marrow fibroblasts (HBMF, osteogenic stem cells, 25 microns in diameter) on the fibre scaffolds for seven days.

Results

All the fibres had very strong electrostatic charge, and adhered to plastic and metal surfaces, particularly tissue culture containers. Charged polymer fibres may help cell signalling, cell attachment, cell growth and tissue formation.

The fibre scaffolds were examined under light and scanning electron microscopes. Three different sizes of fibres were seen for the PCL-65 fibre scaffolds. The diameter of the fine fibres was about 3 microns with a gap size of about 16 microns. The fibres of Eudragit E100 fibre scaffolds appeared to be transparent, and very homogenous. The diameter of the fibres of the Eudragit E100 fibre scaffolds was about 7.5 microns, with a gap size of about 50 to 200 microns. As with Eudragit E100, the fibres of the PMMA fibre scaffolds were very homogenous. The diameter of the fibres was about 10 microns, with a gap size of about 32 microns.

Fibre scaffolds were saturated with culture medium before osteogenic stem cells were seeded. Experimental results showed that the PCL-65 scaffold was about 90% saturated, and cells were able to seed on the scaffold and survive.

FIG. 9 shows a reproduction of a photograph of the Eudragit E100 fibre scaffold taken at 100 times magnification prior to saturation with the culture medium. The microstructure of the Eudragit E100 scaffold appears to be very homogenous (having fibres with a diameter of about 7.5 microns and a fibre gap size of about 50-200 microns) and thus may be appropriate for used as a substrate to support cell attachment and maybe cell movement of cells of this size. However, when the Eudragit E100 scaffold was saturated completely in the medium, the fibre scaffold dissolved in the culture medium, resulting in a very acidic culture environment. It is, however, possible that cross-linking the Eudragit E100 fibre scaffolds may make them insoluble in cell culture medium, and thus suitable for cell culturing.

PMMA is not a biodegradable polymer and the scaffold remained dry after 7 days, rendering it unsuitable for cell culture.

HBMF cells were then seeded onto PCL-65 scaffolds and cultured for 7 days. Half of the scaffolds were stained with toluidine blue for visualisation. The other half of the scaffolds were fixed in 4% formaldehyde/PBS, embedded in an OTC compound and frozen to −30° C. for cryostat sectioning. FIG. 10 shows an image taken at 1000 times magnification of a resultant section. As set out in table 2, the determined fibre diameter was 3 microns and the fibre gap size was about 16 microns.

Conclusions:

Scaffolds generated from PCL-65 are biocompatible with HBMF cells, as the morphology of the cells remained normal and no sign of cytotoxicity was detected.

### EXAMPLE 3

Having shown that PCL-65 fibre scaffolds are biologically compatible (biocompatible)with cells, further PCL-65 fibre scaffolds and fibre scaffolds produced from two other polymers, polylactide (isomer L:D=96:4) and Eudragit RL100, were used for cell culturing, in order to study the interaction of human bone marrow fibroblasts (HBMF, osteogenic stem cells) with the fibre scaffold and to determine the cell morphology.

In this example, the fibre scaffolds were again produced on aluminium foil using the apparatus shown in FIG. 1. The polymer formulations and fibre production parameters are set out in Table 3 below. In this case, the fibre scaffolds had a thickness of about 0.5 mm thick.

TABLE 3

| | | Polylactide | Eudragit |
|---|---|---|---|
| | PCL65 | (isomer L:D = 96:4) | RL100 |
| Polymer formulation | 20% in | 40% in ethyl | 22.5% in |
| | acetone | alcohol (w/v) | acetone |
| | (w/v) | | (w/v) |
| Nozzle to plate distance | 14.5 | 14.5 | 15.5 |
| (cm) | | | |
| Polymer formulation | 10 | 28 | 3 |
| flow rate (ml/hr) | | | |
| Voltage (kV) | 27 | 23 | 20 |
| Fibre diameter (micron) | 3 | 3 | 10 |

The fibre scaffolds were removed from the aluminium foil for cell culturing. The fibre scaffolds were washed in water and soaked in phosphate buffered solution (PBS) overnight and then with cell culture medium. HBMF cells were seeded onto the fibre scaffolds. The HBMF cells were genetically labelled with a green fluorescent protein (GFP), using a nuclei transfer technique. The cells were grown in antibiotic G418 for selection. When the GFP was expressed in cells, it rendered the cells fluorescent and thus easy to visualise by microscopic examination. The cells were grown for 21 days, and examined using light, fluorescence and scanning electron microscopes, and focused ion beam techniques on days 4, 7, 14 and 21. Fibre scaffolds without cells were used as controls.

Results:

HBMF cells were seen to be attached to the fibres of the fibre scaffolds with cell processes stretching along the fibres. FIG. 11 shows a reproduction of a photograph originally taken at 100 times magnification illustrating cell growth of green fluorescent protein-labelled HBMF cells on a PCL-65 fibre scaffold after seven days in cell culture medium while FIG. 12 shows a reproduction of a focussed ion beam scan at 1000 magnification of HBMF cells on a PCL-65 fibre scaffold after seven days in cell culture medium. As can be seen from FIGS. 11 and 12, the cells have a morphology that appears to resemble nerve cells which might suggest cell differentiation was occurring, without the addition of extrinsic biological factors. This may be because the topography of the fibre scaffolds, such as fibre diameter, has an effect on cell phenotype signalling the stem cells to differentiate into another cell type, and to proliferate in preferred growth rates.

Cell growth was found on PCL-65 scaffold, and cell confluence was obtained at day 21. Similar to the cell growth on PCL-65 scaffold, many cells survived on the Eudragit RL100 scaffold. Cell growth was also found on polylactide scaffold, although only a few cells survived. Some dead and fragmented cells were also seen. This suggests that HBMF cells may not be compatible with polylactide (isomer L:D=96:4).

These results show that fibre scaffolds generated from PCL-65 and Eudragit RL100 can be used as a substrate to support cell attachment and maybe cell movement, and that the HBMF cells appeared to prefer the PCL-65 fibre scaffold which may be due to the smaller fibre diameter. HBMF cells did not form abundant extracellular matrix on the scaffolds, and cell confluence was obtained only after 21 days in culture medium, suggesting that the network of the scaffolds, which is dictated by the fibre diameter and gap size, may not be optimal for cell proliferation. Scaffolds with fibre gap sizes of at least 100 microns may be preferable as they should allow cell penetration into the inner part of the fibre scaffold.

In Examples 1 to 3, the fibre scaffolds were seeded with cells. Experiments were also carried out to determine whether it would be possible to use electric field effect technology to spray cells to enable, for example, electric field effect technology rather than seeding to be used to apply cells to the fibre scaffolds.

### EXAMPLE 4

In this example, apparatus similar to that shown in FIG. 1 was used to determine whether electric field effect technology could be used to spray culture medium placed in the reservoir in place of the polymer formulation. In this example, Dulbecco's modified eagle's medium (DMEM) formulated with water-soluble polymer, polyethylene oxide (PEO, molecular weight=100,000) was used. Various different DMEM concentrations of formulation were tested with different spraying parameters (different nozzle to plate or surface area 7 distances, flow rates and voltages) as shown in Table 4 below. The results are summarised in the comments column of table 4.

TABLE 4

| | Nozzle | | | | |
|---|---|---|---|---|---|
| | to plate | | | | |
| Formulation | distance | Flow rate | | Voltage | Comments |
| 0.6 g PEO in | 2 cm | 0.5 | ml/hr | +11 kV | Droplets were formed, |
| 10 ml DMEM | | | | | but the single jet |
| | | | | | was not very stable |
| 0.8 g PEO in | 2 cm | 0.5 | ml/hr | +11 kV | Droplets were formed, |
| 10 ml DMEM | | | | | with very stable |
| | | | | | 1-2 jets. |
| | 2 cm | 0.8 | ml/hr | +11 kV | Droplets were formed, |
| | | | | | with very stable |
| | | | | | 1-2 jets. |
| | 2 cm | 1 | ml/hr | +11 kV | Droplets were formed, |
| | | | | | with unstable multi- |
| | | | | | jets. |
| 1 g PEO in | 2 cm | 0.8 | ml/hr | +11 kV | Droplets were formed |
| 10 ml DMEM | | | | | (some with fibrils), |
| | | | | | with very stable |
| | | | | | multi-jets. |
| | 2 cm | 1 | ml/hr | +11 kV | Droplets were formed |
| | | | | | (some with fibrils), |
| | | | | | with stable multi- |
| | | | | | jets. |
| | 2 cm | 1.2 | ml/hr | +11 kV | Droplets were formed |
| | | | | | (some with fibrils), |
| | | | | | with unstable multi- |
| | | | | | jets. |
| | 2 cm | 1.5 | ml/hr | +11 kV | Droplets were formed |
| | | | | | (some with fibrils), |
| | | | | | with unstable multi- |
| | | | | | jets |
| 1.2 g PEO in | 2 cm | 1 | ml/hr | +11 kV | Droplets were formed |
| 10 ml DMEM | | | | | (some with fibrils), |
| | | | | | with very stable |
| | | | | | multi-jets. |
| 1.4 g PEO in | 2 cm | 1 | ml/hr | +11 kV | <2 microns beaded |
| 10 ml DMEM | | | | | fibres and some |
| | | | | | 70-100 microns |
| | | | | | droplets, with |
| | | | | | unstable multi-jets. |
| | 3 cm | 1 | ml/hr | +22 kV | <2 microns beaded |
| | | | | | fibres and some |
| | | | | | 70-100 microns |
| | | | | | droplets, with |
| | | | | | stable multi-jets. |
| | 3 cm | 2 | ml/hr | +22 kV | <2 microns beaded |
| | | | | | fibres and some |
| | | | | | 70-100 microns |
| | | | | | droplets, with |
| | | | | | stable multi-jets. |
| | 4 cm | 1 | ml/hr | +22 kV | <2 microns beaded |
| | | | | | fibres and some |
| | | | | | 70-100 microns |
| | | | | | droplets, with |
| | | | | | stable multi-jets. |
| | 4 cm | 2 | ml/hr | +22 kV | <2 microns beaded |
| | | | | | fibres and some |
| | | | | | 70-100 microns |
| | | | | | droplets, with |
| | | | | | stable multi-jets. |
| 1.6 g PEO in | 5 cm | 2 | ml/hr | +23 kV | <2 microns fibres |
| 10 ml DMEM | | | | | and some 70-100 |
| | | | | | microns droplets, |
| | | | | | with very stable |
| | | | | | multi-jets. |
| | 6 cm | 2 | ml/hr | +30 kV | <2 microns fibres |
| | | | | | and some 70-100 |
| | | | | | microns droplets, |
| | | | | | with very stable |
| | | | | | multi-jets. |
| | 6 cm | 4 | ml/hr | +30 kV | About 2 microns |
| | | | | | fibres and some |
| | | | | | 70-100 microns |
| | | | | | droplets, with |
| | | | | | unstable multi-jets. |
| 1.8 g PEO in | 5 cm | 2 | ml/hr | +23 kV | <2 microns fibres |
| 10 ml DMEM | | | | | and some 70-100 |
| | | | | | microns droplets, |
| | | | | | with very stable |
| | | | | | multi-jets. |
| | 6 cm | 4 | ml/hr | +30 kV | About 2 microns |
| | | | | | fibres and some |
| | | | | | 70-100 microns |
| | | | | | droplets, with |
| | | | | | unstable multi-jets. |
| 2 g PEO in | 5 cm | 2 | ml/hr | +23 kV | <2 microns fibres |
| 10 ml DMEM | | | | | and some 70-100 |
| | | | | | microns droplets, |
| | | | | | with very stable |
| | | | | | multi-jets. |
| | 6 cm | 4 | ml/hr | +30 kV | About 2 microns |
| | | | | | fibres and some |
| | | | | | 70-100 microns |
| | | | | | droplets, with |
| | | | | | unstable multi-jets. |

Results:

Cell culture medium, DMEM, sprayed as polydispersed droplets when less than 14% (w/v) PEO was present in the medium. As can be seen from the comments column of Table 4, when the percentage of PEO in the medium was greater than or equal to 14%, fibres of diameter of about 2 microns and droplets of 70-100 microns were formed. No other additive was required, not even surfactant.

These results indicate that the presence of water-soluble polymer such as PEO, PVP (polyvinyl pyrrolidone) and PVA (poly vinyl alcohol) may enable such aqueous formulations to be sprayed using an electric field effect technology process.

### EXAMPLE 5

Following on from Example 4, further experiments were carried with starch corn added to the PEO/DMEM formulation to mimic the presence of biological material or cells. The amount of starch corn and percentage of polymer present in the culture media and spraying parameters are set out in Table 5. The results are summarised in the comments column of Table 5.

TABLE 5

| | Nozzle | | | | |
|---|---|---|---|---|---|
| | to plate | | | | |
| Formulation | distance | Flow rate | | Voltage | Comments |
| 0.1 g starch | 5.5 cm | 2 | ml/hr | +30 kV | Droplets and starch |
| corn in 5 ml | | | | | corn were seen, with |
| 12% PEO/ | | | | | very stable mutli-jets |
| medium | | | | | |
| 0.1 g starch | 5.5 cm | 2 | ml/hr | +30 kV | About 2 microns fibres |
| corn in 5 ml | | | | | and some 70-100 |
| 20% PEO/ | | | | | microns droplets were |
| medium | | | | | formed, with very |
| | | | | | stable multi-jets. Some |
| | | | | | starch corn was |
| | | | | | incorporated into the |
| | | | | | fibres. |
| 0.1 g starch | 5.5 cm | 2.5 | ml/hr | +30 kV | About 2 microns fibres |
| corn in 5 ml | | | | | and some 70-100 |
| 25% PEO/ | | | | | microns droplets were |
| medium | | | | | formed, with stable |
| | | | | | multi-jets. Some starch |
| | | | | | corn was incorporated |
| | | | | | into the fibres. |

Results:

Starch corn of about 10 microns in diameter was seen, together with droplets or fibres of the formulations used, depending on the percentage of water soluble polymer (in the examples given PEO), present in the formulations, indicating that it may be possible to spray biological material and cells using this technique.

### EXAMPLE 6

The fibre scaffolds described above were produced from polymer solutions with the solvent evaporating in ambient air during the fibre production. In some circumstances, however, the solvents available for a polymer may not be compatible with the cells to be seeded or sprayed on the fibre scaffold. Also, certain types of polymers such as poly(3-hydroxybutyric acid) (Biopol), require the use of toxic solvents such as methylene chloride. In light of this, further experiments were carried out to determine whether the fibre scaffolds could be produced from molten polymer.

In these experiments, polymers, for example, polycaprolactone (PCL, 65,00) were melted and moulded to form solid sticks of 1.2 cm diameter and 20 cm in length. The polymer sticks were inserted into an inlet tube of a hot gas gun, so that one end of the stick was in direct contact with a heating element. The temperature of the heating element was constantly maintained, in this example 204° C., by combusting butane gas. The outlet of the gun was a metal nozzle, which was close to the heating element. A syringe pump was directly attached to the other end of the polymer stick so that the stick was pressed downwards by the pump and the flow rate of the molten polymer at the nozzle could be adjusted accordingly using the syringe pump. The hot gas gun was positioned in such a way that the nozzle was pointing vertically downwards. An electric field was generated by connecting the end of the nozzle to a high voltage generator and locating an earthed (grounded) plate beneath the nozzle to form the surface area 7. Molten polymer issuing from the nozzle formed an electrically charged jet which, when the molten polymer was allowed to cool and solidify in ambient air, solidified to form a fibre which deposited onto the plate.

PCL-65 was melted and sprayed using this arrangement and using different fibre production parameters (flow rate, voltage and nozzle to earthed plate distance). In each case, a single electrically charged polymer jet was produced which solidified to form fibre which was collected on the earthed plate, as a continuous web of fibre.

Table 6 below shows the results achieved with certain sets of fibre production parameters. The fibre diameter of the resultant fibre scaffold depended upon the fibre production parameters as set out in Table 6 being, for a fixed distance between the nozzle and the earthed surface, dependent on the flow rate of the molten polymers. Depending upon the fibre production parameters, the fibre diameter lay in the range in size from 20-70 microns. The fibre gap size was in the range of 100-500 microns.

TABLE 6

| Flow rate | Voltage | Distance between nozzle | Fibre diameter |
|---|---|---|---|
| (ml/hr) | (kV) | and earthed plate (cm) | (microns) |
| 4 | 23 | 25 | 50 |
| 5 | 22 | 25 | 60 |
| 2.5 | 16.5 | 22 | 70 |
| 2.5 | 10 | 23 | 70* |

*In this example, the fibre was collected on an earthed rotating metal rod with a diameter of about 1 cm (centimetre).

Macroporous fibrous scaffolds may thus be generated, for example by spraying molten polymer, to create fibre scaffolds that resemble bone, ligament, cartilage or tendon-like structures for culturing cells, such as osteogenic or progenitor cells in order to create bone, ligament, cartilage and tendon tissues.

As described above, fibre scaffolds can be produced that can be used for culturing of mammalian, including human, cells so that biological tissues can be produced engineered or produced artificially. Examples of such cells that may be used for cell culture are skin fibroblasts, osteogenic cells, progenitor cells, muscle cells and bone marrow stem cells. Thus artificial or tissue engineered biological material such as skin, bone, ligament, cartilage, muscle and tendon may be formed.

There is also potential for tissue regeneration from cells with stem cell characteristics. The development of osteoblasts, chrondroblasts, adipoblasts, myoblasts and fibroblasts results from colonies derived from such single cells. They may, therefore, be useful for regeneration of all tissues that this variety of cells comprises: bone, cartilage, fat, muscle, tendons and ligaments.

Fibre scaffolds of fibre diameter such as 25 microns and gap size, for example, 150-200 microns may be suitable for stem cell and/or differentiated cell attachment, movement, differentiation, proliferation and formation of extra cellular matrices.

Regeneration of tissues may also be enhanced by combining the principal of gene therapy with tissue engineering. This could be achieved by spraying, for example using the electric field effect technology or other suitable technique, a plasmid DNA carrying the gene for a protein or growth factor on to the fibre scaffold, or by incorporating plasmid DNA into the fibre scaffold polymer formulation so that the fibre scaffold production results in plasmid DNA being physically entrapped within the fibre scaffold. The plasmid DNA may also carry a promoter/repressor gene so that the expression of the gene for the protein/growth factor can be turned on or off as desired. Fibre scaffolds containing plasmid DNA may enhance cell attachment and proliferation, and regeneration of tissues.

Mammalian cells/platelets, may be sprayed using electric field effect technology to enable the delivery of live cells to wounded or defective tissues such as skin, bone, cartilage, tendon and cornea. Also, biological micro-organisms, healthy cells, cultured cells or genetically engineering cells that express a therapeutic protein, may be sprayed directly onto a target area, such as skin, bone, cartilage, wounds and burns, for cell or gene therapy.

The fibre scaffolds may be provided on or in a wound incorporated or implanted into a body, for supporting tissue growth, such as skin, bone, muscle, fat, ligament, cartilage and tendon. The fibre scaffolds may be produced in vitro or in situ, that is directly at a target area of the mammalian body such as a wound, injury or other area where tissue regeneration is required. Where the fibre scaffold is formed in situ, then surface area 7 shown in FIGS. 1 and 2 will be the target area of the mammalian body to which the fibre scaffold is to be applied. Biological tissue generated in vitro using such fibre scaffolds may be used for transplantation in wounds, dermal burns, bone fractures or cartilage degeneration.

Although the fibre scaffolds described above and shown in FIGS. 8 to 11 are formed on flat surface areas, this need not necessarily be the case. For example, the fibre scaffolds may be deposited onto curved surface areas and may be cut or otherwise formed into a desired shape. Also, tubular fibre scaffolds may be formed by, for example, using a rotating mandrel as the surface area.

Active ingredients such as drugs or medicaments that do not affect or enhance cell growth may be sprayed onto the polymer fibre scaffolds described above or may be incorporated in the polymer fibre for controlled released delivery. Other active ingredients may be sprayed onto the polymer fibre scaffolds or may be incorporated in the polymer fibre for controlled released delivery including biological micro-organisms, healthy cells, cultured cells or genetically engineering cells that express a therapeutic protein, proteins, enzymes, for enzyme or hormone therapy, drugs or other medicaments. When cells are encapsulated into the polymer fibres, they might be protected from immunological processes, and may thus survive and maintain an effective supply of proteins, and therefore may be useful in enzyme or hormone therapy. Blood vessel cells such as endothelial cells may delivered to a fibre scaffold or an injury site to promote neo-vascularisation and thus enhance the healing process. When blood clot formation cells such as platelets are delivered to a bleeding area, further blood loss could be prevented.

From a tissue engineering point of view, as described above cells could either be sprayed (using electric field technology or another suitable spraying process) or seeded to migrate into the fibre scaffolds, where they undergo cell proliferation and differentiation. Spraying of cells onto the fibre scaffold may be accomplished using a separate electric field effect apparatus or possibly by providing separate reservoirs and outlets for the polymer formulation and cell formulation in the same apparatus, for example, along the lines shown in FIG. 11 of WO98/03267. Cells may be sprayed using an opposite polarity voltage from that used for the fibre scaffold production to facilitate deposition on the fibre scaffolds.

The fibre scaffolds may be sprayed or seeded with genetically engineered cells that carry a plasmid DNA with a promoter/represser gene (so that the level of expression of a protein can be controlled), before implantation to an injury site. By including a fibre scaffold, the level and duration of transgene expression by implanted cells may be enhanced.

As described above, the fibre scaffolds are each formed of a single type of polymer, the composition of the fibre polymer may be varied through the fibre scaffold and fibre scaffolds having of regions of different fibre diameter and/or gap size may be provided. Also, more than one type of polymer or more than one type of polymer fibre may be incorporated in a fibre scaffold.

Reference is made above to fibre diameter. It is however possible that at least in some circumstances the fibres may not be precisely circular in cross-section. In such cases, the fibre diameter should be taken as meaning the width of the fibre as viewed through the microscope, that is viewed from the surface of the fibre scaffold.

1. A method of enabling growth of mammalian cells, which method comprises: supplying liquid comprising biologically compatible polymer to a liquid outlet in the vicinity of a surface and subjecting liquid issuing from the outlet to an electric field to cause the liquid to form polymer fibre which is attracted to and deposits onto the surface to form a polymer fibre scaffold having fibre of a given diameter with gaps between adjacent fibre portions; and applying mammalian cells to the fibre scaffold, wherein the gaps between the fibre portions and the fibre diameter have a size relative to a diameter of the mammalian cells such that cells grow or elongate preferentially along the fibre of the fibre scaffold.

2. A method according to claim 1, wherein the fibre diameter is comparable to or smaller than the cell diameter.

3. A method according to claim 1, wherein the cell diameter is from 1 to 20 times the fibre diameter.

4. A method according to claim 1, wherein the cell diameter is from 5 to 10 times greater than the fibre diameter.

5. A method according to claim 1, wherein the cell diameter is in the range from about 2 to about 20 microns and the fibre diameter is in the range from about 1 to 2 microns.

6. A method according to claim 1, wherein the cell diameter is about 10 microns and the fibre diameter is from 1 to 2 microns.

7. A method according to claim 1, wherein the fibre diameter is from 1 to 2 microns.

8. A method according to claim 1, wherein the relative sizes of the cell and fibre diameters are such that the fibre surface appears curved to the cells.

9. A method according to claim 1, wherein the fibre diameter is of comparable size to cell surface receptors of the cells.

10. A method according to claim 1, wherein the polymer is selected from the group consisting of New Skin, Eudragit RL100, polycaprolactone, polylactide (L:D isomer ratio 50:50) and polylactide (L:D isomer ratio 96:4).

11. A method according to claim 1, wherein the cells are human adherent cells.

12. A method according to claim 1, wherein the cells are human fibroblast cells.

13. A method according to claim 1 wherein the mammalian cells include human fibroblast cells, and the polymer fibre scaffold has fibre of diameter in a range of 1 to 2 microns with gaps between adjacent fibre portions.

14. A method of facilitating at least one cell process of human fibroblast cells, which method comprises: supplying liquid comprising a biologically compatible polymer selected from the group consisting of New Skin, Eudragit RL100, polycaprolactone and polylactide to a liquid outlet in the vicinity of a surface and subjecting liquid issuing from the outlet to an electric field to cause the liquid to form polymer fibre which is attracted to and deposits onto the surface to form a polymer fibre scaffold having fibre of a diameter in a range of 1 to 2 microns with gaps between adjacent fibre portions; and applying the human fibroblast cells to the fibre scaffold, wherein the gaps between the fibre portions and the fibre diameter are such that the human fibroblast cells grow or elongate preferentially along the fibre of the fibre scaffold.

15. A method according to claim 20 wherein the mammalian cells comprise human bone marrow fibroblast cells, and wherein the mean fibre diameter of fibres in the a polymer fibre scaffold is about 3 microns with the mean size of gaps between adjacent fibre portions of about 16 microns.

16. A method of providing an environment for facilitating differentiation of stem cells, which method comprises: supplying liquid comprising a biologically compatible polymer to a liquid outlet in the vicinity of a surface and subjecting liquid issuing from the outlet to an electric field to cause the liquid to form polymer fibre which is attracted to and deposits onto the substrate to form a polymer fibre scaffold having fibre of diameter that, without addition of extrinsic biological factors, facilitates differentiation.

17. A method according to claim 16, further comprising applying stem cells to the fibre scaffold without addition of extrinsic biological factors.

18. A method of facilitating differentiation of osteogenic stem cells, which method comprises: supplying liquid comprising a biologically compatible polymer to a liquid outlet in the vicinity of a surface and subjecting liquid issuing from the outlet to an electric field to cause the liquid to form polymer fibre which is attracted to and deposits onto the substrate to form a polymer fibre scaffold having fibre of diameter of about 10 microns with gaps between adjacent fibre portions of about 16 microns; and applying the cells to the fibre scaffold without addition of extrinsic biological factors but wherein, after a period of time, the cells have a morphology resembling nerve cells.

19. A method according to claim 16, wherein the polymer comprises polycaprolactone.

20. A method of facilitating at least one cell process of mammalian cells, which method comprises: supplying liquid comprising a solution of a biologically compatible polymer to a liquid outlet in the vicinity of a surface and subjecting liquid issuing from the outlet to an electric field to cause the liquid to form polymer fibre which is attracted to and deposits onto the substrate to form a polymer fibre scaffold having fibre of a diameter in the range from 0.2 to 100 microns with gaps between adjacent fibre portions in the range from about 10 to 500 microns; and applying mammalian cells to the fibre scaffold.

21. A method of facilitating at least one cell process of mammalian cells, which method comprises: supplying liquid comprising a biologically compatible polymer melt to a liquid outlet in the vicinity of a surface and subjecting liquid issuing from the outlet to an electric field to cause the liquid to form polymer fibre which is attracted to and deposits onto the substrate to form a polymer fibre scaffold having fibre of a diameter in the range from 2 to 500 microns with gaps between adjacent fibre portions in the range from about 25 to 3000 microns; and applying mammalian cells to the fibre scaffold.

22. A method according to claim 1, wherein the polymer formulation is a polymer solution.

23. A method according to claim 1, wherein the polymer formulation is a polymer melt.

24. A method of forming a fibre scaffold for facilitating at least one cell process of mammalian cells, which method comprises: supplying comprising biologically compatible molten or liquid polymer to a liquid outlet in the vicinity of a surface and subjecting liquid issuing from the outlet to an electric field to cause the liquid to form polymer fibre which is attracted to and deposits onto the substrate to form a polymer fibre scaffold having fibre of a diameter in the range of from 20 to 70 microns and a gap size between adjacent fibre portions in the range of 100 to 500 microns.

25. A method according to claim 24, wherein the fibre scaffold is arranged to be implanted in a mammalian body or placed on or in a wound.

26. A method according to claim 24, wherein the surface is a target area of a mammalian body such as a wound and the fibre scaffold is produced in situ.

27. A method according to claim 1 wherein the cells are applied by a seeding process.

28. A method according to claim 1 wherein the cells are applied by spraying.

29. A method according to claim 1 which comprises preparing a liquid formulation suitable for enabling cells to be applied to the fibre scaffold by subjecting the liquid formulation to an electric field to cause the liquid to break up into droplets, which comprises formulating cell culture medium with a water soluble polymer.

30. A method according to claim 1 which comprises applying the cells to the fibre scaffold by subjecting a liquid formulation comprising cell culture medium carrying the cells and a water soluble polymer to an electric field to cause the liquid to break up into droplets or to form at least one fibre.

31. A method of applying cells to a substrate, which method comprises subjecting a liquid formulation comprising cell culture medium carrying the cells and a water soluble polymer to an electric field to cause the liquid to break up into droplets or to form at least one fibre.

32. A method according to claim 31, wherein the water-soluble polymer is selected from the group consisting of PEO, PVP and PVA.

33. A method according to claim 32, wherein the cell culture medium is DMEM.

34. A method of forming a polymer fibre scaffold, for example to form a wound dressing, which method comprises producing polymer fibre using electric field effect techniques so that the polymer fibre deposits onto the surface of a target area, such as skin and/or wound, to form a covering or dressing for the target area, wherein the polymer fibre production is controlled to control the polymer charge and relaxation time, and thereby control the lateral force experienced by the polymer fibre resulting from the fibre that has already settled on the target area, so as to control the pattern of deposition of the polymer fibre on the target area, to produce a lattice or web like polymer fibre scaffold to facilitate the formation of skin tissue by fibroblasts of a weave pattern rather than an aligned parallel pattern.

35. A method according to claim 1 wherein the fibre gap is greater than approximately half the cell diameter.

36. A method according to claim 1 wherein the fibre diameter is less than the fibre gap.

37. Apparatus for enabling growth of mammalian cells, which method comprises: a reservoir of liquid comprising biologically compatible polymer having a liquid outlet; an electric field generator configured to generate an electric field in a region between the liquid outlet and a substrate to cause liquid issuing from the outlet to form polymer fibre which is attracted to and deposits onto the substrate to form a polymer fibre scaffold having fibre of a given diameter with gaps between adjacent fibre portions; and a cell applier for applying mammalian cells to the fibre; and an applier for applying cells to the fibre scaffold, wherein the apparatus is configured to control the size of the gaps between the fibre portions and the fibre diameter such that the cells grow or elongate preferentially along the fibre of the fibre scaffold.

38. Apparatus according to claim 37, wherein the apparatus is configured to control the fibre diameter to be comparable to or smaller than the cell diameter.

39. Apparatus according to claim 37, wherein the apparatus is configured to control the fibre diameter such that the cell diameter is from 1 to 20 times the fibre diameter.

40. Apparatus according to claim 37, wherein the apparatus is configured to control the fibre diameter such that the cell diameter is from 5 to 10 times greater than the fibre diameter.

41. Apparatus according to claim 37, wherein the applier is configured to provide cells having a cell diameter in the range from about 2 to about 20 microns and the apparatus is configured to control the fibre diameter to be in the range from about 1 to 2 microns.

42. Apparatus according to claim 37, wherein the applier is configured to provide cells having a cell diameter of about 10 microns and the apparatus is configured to control the fibre diameter to be from 1 to 2 microns.

43. Apparatus according to claim 37 wherein the polymer is selected from the group consisting of New Skin, Eudragit RL100, polycaprolactone, polylactide (L:D isomer ratio 50:50) and polylactide (L:D isomer ratio 96:4).

44. Apparatus according to any 43, claim 37 wherein the polymer formulation is a polymer solution.

45. Apparatus according to any 43, claim 37 further comprises a heater for melting polymer to provide the polymer formulation.

46. Apparatus according to 43, claim 37 wherein the applier comprises human cells such as fibroblast cells or stem cells.

47. Apparatus according to 16, claim 37 wherein the fibre gap is greater than approximately half the cell diameter.

48. Apparatus according to 47, claim 37 wherein the fibre diameter is less than the fibre gap.

49. A method according to claim 1, wherein the surface is a target area of a mammalian body such as a wound and the fibre scaffold is produced in situ.

50. A method according to claim 16 wherein the cells are applied by a seeding process.

51. A method according to claim 16 wherein the cells are applied by spraying.
